(19) United States
(12) Patent Application Publication
(10) Pub. No.: US 2007/0042402 A1
(43) Pub. Date: Feb. 22, 2007

(54) Method for Predicting Human Longevity

(76) Inventors: Thomas E. Johnson, Louisville, CO (US); Shane L. Rea, Boulder, CO (US); Deqing Wu, Boulder, CO (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(21) Appl. No.: 11/459,293

(22) Filed: Jul. 21, 2006

Related U.S. Application Data
(60) Provisional application No. 60/701,244, filed on Jul. 21, 2005.
(60) Provisional application No. 60/758,441, filed on Jan. 11, 2006.
(60) Provisional application No. 60/758,411, filed on Jan. 13, 2006.

(51) Int. Cl. C12Q 1/68

(52) U.S. Cl. 435/6

(57) ABSTRACT

The present disclosure concerns methods of detecting levels of one or more molecules(s), longevity predicting marker(s), in a sample, and using these levels of the marker(s) to predict a condition. In a particular embodiment, the present invention concerns methods of detecting longevity predicting marker(s), in a sample, preferably to assess the overall health status in a subject predisposed to a condition. In certain embodiments, the methods may comprise obtaining a sample from a subject such as a blood, saliva or buccal swab sample and analyzing the sample for the presence or level of a longevity predicting marker(s) to predict a condition. In another embodiment, the methods may comprise analyzing a sample from a subject for the presence of longevity predicting marker(s) and applying information obtained from analyzing the presence of longevity predicting marker(s) to determine a treatment for a medical condition of the subject.

### CROSS-REFERENCE TO RELATED APPLICATIONS

The present application claims the benefit under 35 U.S.C. §119(e) of provisional U.S. patent applications Ser. No. 60/701,244 filed on Jul. 21, 2005 and Serial No. 60/758,411 filed on Jan. 11, 2006.

### FEDERALLY FUNDED RESEARCH

The studies disclosed herein were supported in part by grants P01 AG08761 and R01 AG016219 from the National Institutes of Health. The U.S. government may have certain rights to practice the subject invention.

### FIELD

The present invention relates to methods for detection of longevity predicting marker(s) in a sample for use in diagnosis of overall health of a subject. In certain embodiments, the disclosed methods may be used to identify the presence or onset of a condition in a subject. Various embodiments of the present invention concerns methods for predicting longevity, life span and/or beneficial interventions in subjects. In some embodiments, the subjects may be biowarfare agents such as bacteria or other microorganisms. The disclosed methods are of use to assess an individual's health, particularly cardiac health and if needed to intervene with an appropriate treatment.

### BACKGROUND

Researcher have long attempted to formulate tests to predict overall health and or longevity in organisms including humans. A Biomarker of Aging (BoA) is a biological parameter of an organism that either alone or in some multivariate composite will, in the absence of disease, better predict functional capability at some late age, than will chronological age. Many BoAs have been studied such as human studies by the National Institute on Aging. Despite the obvious need for BoAs to provide assessments for pharmacological and other interventions into aging, few if any BoAs have been successfully developed. The American Federation for Aging Research proposes as criteria for a BoA: A BoA typically predicts the rate of aging (e.g. indicates where a person is in their total life span) and is typically a better predictor of life span than chronological age. In addition, a BoA typically monitors a basic process that underlies the aging process, not the effects of disease. Another, typical criteria for a BoA is that the tests for the BoA are repeatable and do not harm the subject. Last, a BoA typically is something that works in humans and in laboratory animals, such as mice, to facilitate testing in lab animals before being validated in humans.

Chance plays a large and probably ineradicable role in determining variation among individuals in age at death. In humans, as well as populations of laboratory animals, 60-90% of the variation in age at death is independent of genotype. In isogenic populations (where genetic variance is essentially zero), under a uniform environment, some individuals die early in life and others live quite long. Differences in individual life span of Caenorhabditis elegans can reach as much as 50-fold and still have almost as much variation in time of death as does the population of the United States. Such observations make suspect the popular notion of a “genetic program that regulates longevity.” Instead, geriatric, demographic and evolutionary evidence suggest an alternate paradigm of aging; one that encompasses a rich variety of often highly plastic processes, influenced by genetic, environmental, and stochastic phenomenon. It has previously been demonstrated that the ability of individual isogenic worms to respond to stress on the first day of adult life has a large stochastic component and is a major predictor of their subsequent longevity. A reporter based on the promoter of the alpha-B crystallin-like protein, hsp-16.2 was utilized in such studies. Similar proteins exist in higher organisms and expression of these molecules likely reflect on the health and/or longevity in these organisms.

Although BoAs have been sought after for over 15 years, very few have been discovered and even have become of use. A need exists for the finding and use of BoA's to predict overall health of an individual or the need for early intervention of a condition implicated by the level of these agents.

### SUMMARY

The present invention concerns methods and compositions for predicting longevity, health span and/or beneficial interventions in subjects. In preferred embodiments, the subjects are humans. In other preferred embodiments, the subjects are animals, including but not limited to cats, dogs, horses, cows, goats, pigs, sheep, fish or alpacas. In some embodiments, the subjects may be biowarfare agents such as bacteria or other microorganisms.

The present invention relates to methods for evaluating the level of a longevity predicting marker in a sample to assess a condition. In an exemplary longevity predicting marker assay, a sample from a subject is obtained, one or more longevity predicting marker is detected and based on this information the condition of a subject is evaluated. A longevity predicting marker molecule concentration is assessed in the sample, preferably using an antibody assay, to evaluate the presence or absence or level of longevity predicting marker at a given time. This information may then be used to analyze the condition of a subject, for example the status of an organ such as the heart, kidneys or lungs. In particular embodiments, the longevity predicting marker (s) can be detected in a sample using an enzyme-linked assay (ELISA) directed to a form of a longevity predicting marker. From such analysis, the propensity for a condition, such as a heart condition or cancerous condition may be determined using the information obtained on the concentration of longevity predicting marker in a sample.

In another embodiment, at least one sample from a subject admitted to the intensive care unit may be obtained. In accordance with this embodiment, the sample(s) may be analyzed for one or more longevity predicting marker(s) and the subject is evaluated based on the information obtained on the longevity-predicting marker. In certain embodiments, blood, urine or buccal samples at different times from a subject admitted to the intensive care unit may be obtained and analyzed for the levels of longevity-predicting marker(s). In one example, multiple parameters of an admitted subject, such as age, medical history and gender, will be examined in combination with measuring the longevity-predicting marker levels to assess the survivability of a subject. In addition, this criteria will be used to assess treatment of the subject with at least one therapeutic agent.

In particular embodiments, the methods may include determining the level of a longevity-predicting marker, such as an alpha-B crystallin or HSP27. In accordance with these embodiments, longevity-predicting markers may include HSP27, HSP27 derivatives, HSP27 analogs, alpha-B crystallin, alpha-B crystallin-like or combination thereof. Alternatively, these longevity-predicting markers may include a stress response factor, a stress response protein, CRP (C-reactive protein), an interleukin or a cytokine, in a sample from the subject. In accordance with these embodiments, the predicted longevity of a subject may be determined from the level of the marker(s) present in the sample. Samples may be comprised of urine, blood, tissue, saliva, amniotic fluid, cerebrospinal fluid, fecal matter, exhaled breath or tears or combination thereof.

### BRIEF DESCRIPTION OF THE DRAWINGS

The following drawings form part of the present specification and are included to further demonstrate certain embodiments of the present invention. The embodiments may be better understood by reference to one or more of these drawings in combination with the detailed description of specific embodiments presented herein.

FIG. 1. represents an exemplary schematic of a construct of a Caenorhabditis elegans longevity-predicting marker and a reporter gene.

FIG. 2. represents an exemplary schematic of the stochastic effects on longevity using a Caenorhabditis elegans longevity-predicting marker and a reporter gene.

FIG. 3. represents an exemplary timeline of construct expression in C. elegans before and after stress induction.

FIG. 4. represents one exemplary method for analyzing the expression of a longevity-predicting marker:reporter construct after stress induction.

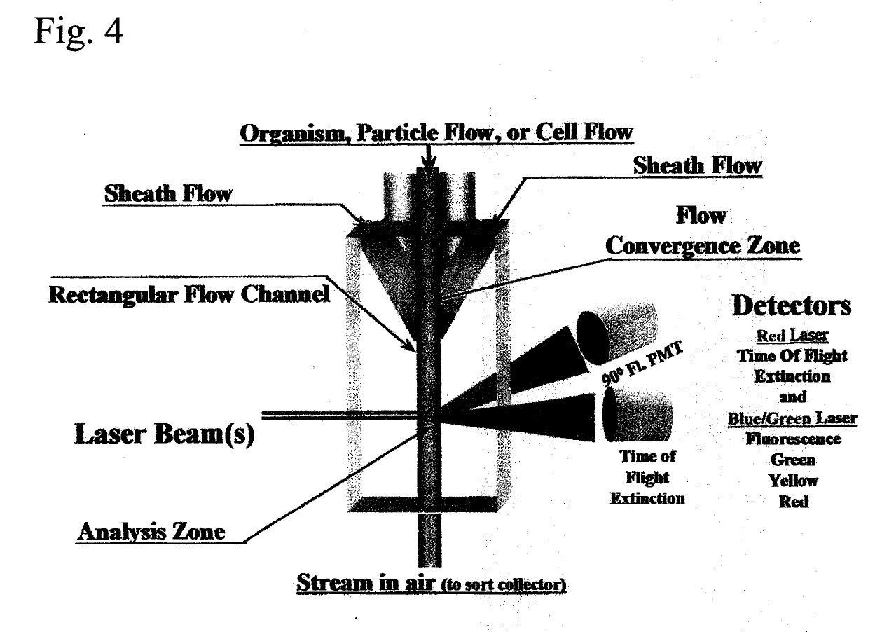

FIG. 5 represents an exemplary method of analysis of survival and thermotolerance of C. elegans previously sorted on differential longevity-predicting marker:reporter expression after stress induction (e.g. 2 hours of heat shock).

FIG. 6 represents an exemplary method of analysis of progeny of C. elegans from parent expression of high or low levels of reporter expression, this exemplary method illustrates that the level of reporter expression is not heritable.

FIG. 7 represents an exemplary method demonstrating survival trajectories of C. elegans in organisms having low, medium and high levels of expression of a longevity-predicting marker:reporter construct.

### DESCRIPTION OF ILLUSTRATIVE EMBODIMENTS

All documents, or portions of documents, cited in this application, including but not limited to patents, patent applications, articles, books, and treatises, are hereby expressly incorporated by reference in their entirety.

Definitions

As used herein, “a” or “an” may mean one or more than one of an item.

As used herein, the terms “and” and “or” may be used to mean either the conjunctive or disjunctive. That is, both terms should be understood as equivalent to “and/or” unless otherwise stated.

Description

In the following sections, various exemplary compositions and methods are described in order to detail various embodiments of the invention. It will be obvious to one skilled in the art that practicing the various embodiments does not require the employment of all or even some of the specific details outlined herein, but rather that concentrations, times and other specific details may be modified through routine experimentation. In some cases, well known methods or components have not been included in the description.

In some embodiments, methods of use of a longevity marker may include assessing the health condition or longevity of a subject by analyzing the level of at least one longevity-predicting marker of a sample from the subject. In accordance with these embodiments, the predicted longevity of a subject may be determined from the level of the marker(s) present in the sample. Samples may be comprised of urine, blood, tissue, saliva, amniotic fluid, cerebrospinal fluid, tears or combination thereof. Other samples may include whole organisms such as yeast, virus or bacteria.

In certain embodiments, the disclosed methods and compositions may be of use to predict subsequent health span. In other embodiments, the disclosed methods and compositions may be of use to make a subject more robust. For example, the methods and compositions may be of use to predict or to manipulate the productivity of animals raised for consumption.

In still other embodiments, the disclosed methods and compositions may be used to predict what interventions may be beneficial to improve longevity, health and/or robustness of a subject. The disclosed methods can be performed without affecting the health or viability of the subject.

In some embodiments, the level of the longevity-predicting marker may be determined by assaying for levels of the molecule the sample. The molecules can include proteins, peptides, or nucleic acid molecules such as mRNA. Such techniques are well known in the art. These techniques include, but are not limited to, antibody or aptamer binding to the marker protein, hybridization of marker mRNA or cDNA to a microarray, sequence specific probe hybridization, sequence specific amplification and similar methods. Any such technique known in the art for determining the expression level of a marker may be used.

In alternative embodiments, the level of one or more longevity-predicting marker can be determined by assaying for metabolic products that reflect the level of a marker. A wide variety of metabolic products in subjects are known and any such known product may be utilized in the claimed methods and compositions. Any intermediate or product of cell metabolism whose concentration reflects the level or activity of a longevity-predicting marker would be of use in the claimed methods. The skilled artisan will realize that in some cases metabolic profiles, or combinations of metabolic intermediates and/or products, may be determined. Such profiles or combinations may provide a more accurate indication of the level of a longevity-predicting marker.

In other embodiments, the expression level of the longevity-predicting marker in the sample may be determined indirectly, for example by using a reporter gene construct linked to a promoter that mimics or duplicates a promoter for an endogenous longevity-predicting marker. The reporter construct may be transformed into isolated cells from the subject. The skilled artisan will realize that although the green fluorescent protein (GFP) was used in the exemplary embodiments incorporated herein, the type of gene used is not limiting and any type reporter gene known in the art may be used. In one embodiment, the expression level of the longevity-predicting marker of a bacteria may be determined. For example, it may be necessary to assess the vulnerability of a bacterial agent to a specific anti-bacterial treatment. A sample of bacteria such as, Escherichia coli, Neisseria gonorrhoeae, Neisseria meningitidis, Pneumococcus spp., Mycobacterium leprae, Brucella abortus, Pseudomonas aeruginosa, Mycobacterium tuberculosis, Mycoplasma pneumonia or combination thereof may be obtained and the level of longevity-predicting marker assessed before and after a treatment with an anti-bacterial agent. Thus, an anti-bacterial, anti-viral, or anti-fungal agent can be tested for effectiveness against a certain organism. For example, if the levels of longevity-predicting marker in the organism decrease after treatment with an agent, this could predict the effectiveness of that agent on that organism. This information may be used to assess a treatment of a subject having been infected with the organism

General Considerations for Early Longevity-Predicting Marker Detection

In one embodiment, the present invention concerns the role of detecting one or more longevity-predicting marker(s) in a sample of a subject for prediction of the presence or onset of a health condition and intervention of its progression. It was demonstrated that HSP16 in nematodes predicts the longevity of the nematode. It is suggested that molecules similar to HSP16 exist in higher organisms and behave similarly to HSP16. These molecules (see Table 1) are referred to as alpha-B crystalline, alpha-B crystallin-like, alpha-A crystalline, alpha-A crystalline-like molecules. Examples of alpha-B crystallin-like molecules include, but are not limited to, HSP27 and HSPB1 thru HSPB10.

Uses of a Longevity-Predicting Marker Assay

Evaluating and Monitoring the Levels of Longevity Predicting Marker

Whether or not the health of a subject can be improved or stabilized may depend, in part, upon the early introduction of therapeutically relevant treatments. In order to eliminate, minimize or attenuate a condition in an individual, it would be helpful to predict these events earlier in progression rather than later. By comparing the individual's specific level of at least one longevity-predicting marker to a normal healthy control, or within a given individual over time, a healthcare provider could determine whether the patient needs to be treated immediately or otherwise observed for a period of time and potentially rechecked for these levels.

Under conditions when a longevity-predicting marker level is measured in a sample of a subject, such as during a routine physical or in a subject predisposed to a condition, it becomes critical that the treating healthcare provider have reliable information available about an individual's concentration of longevity-predicting marker in the sample. For example, a high concentration of longevity-predicting marker (e.g. a reliably significant increase over the average population) is especially likely when a subject is healthy. Alternatively, a low concentration of longevity-predicting marker (e.g. a reliably significant decrease over the average population) is especially likely when a subject is predisposed to a condition such as a heart condition. When a subject presents with low levels of a longevity-predicting marker, a healthcare professional may intervene by performing additional tests on a subject to assess the particular condition such as the presence of a tumor or heart condition. In accordance with this example, a healthcare provider can administer a therapeutic treatment to attenuate the condition to avoid the possibility of permanent damage or death of the patient. In addition, a healthcare professional may monitor the therapeutic treatment of the subject by obtaining samples from the patient after treatment and analyzing the levels of longevity-predicting marker(s) in the sample and assessing the condition of the subject based on these findings. Therapeutic treatments may be altered depending on the concentration of longevity-predicting marker(s) present in the post-treatment sample.

It is contemplated herein that any form of longevity-predicting markers disclosed in the present invention may be assessed for presence or concentration in a sample (see for example Table 1). For example, other alleles or other structural variants of longevity-predicting markers (either endogenous or synthetic) such as HSP27, HSP5, HSP8 or other alpha B- or alpha A-crystallin-like molecules are contemplated as target molecules where the concentration of the molecule can be correlated to overall health of a subject and/or the presence or prediction of a condition.

TABLE 1

| | | E-value | | |
|---|---|---|---|---|
| | | (vs HSP- | Tissue | |
| Gene | Synonym | 16.2)* | Distribution | Comments*** |
| CRYAB | α-B | 2 × 10⁻¹² | Cardiac, | [OMIM: |
| | crystallin, | | skeletal, | 123590]: |
| | HSPB5 | | and smooth | Phenotypes of |
| | | | muscle (and | [CRYAB]: |
| | | | varying | Cataract, |
| | | | amounts other | posterior |
| | | | tissues | polar 2; |
| | | | including | Myopathy, |
| | | | kidney and | cardioskeletal, |
| | | | brain) | desmin-related, |
| | | | | with cataract |
| | | | | (cross-ref. |
| | | | | OMIM: 608810). |
| HSPB8 | CRYAC | 7 × 10⁻¹² | Muscles, | [OMIM: |
| | | | breast (in | 608014]: |
| | | | MCF-7 cells | Phenotypes of |
| | | | HSPB8 is | [HSPB8]: |
| | | | estrogen | Neuropathy, |
| | | | regulated), | distal |
| | | | placenta, | hereditary |
| | | | prostate, | motor, type II |
| | | | colon, | (cross-ref. |
| | | | brain, and | OMIM: 158590). |
| | | | keratinocytes | |
| CRYAA | α-A | 7 × 10⁻¹⁰ | Eye lens | [OMIM: |
| | crystallin, | | | 123580]: |
| | HSPB4 | | | Phenotypes of |
| | | | | [CRYAA]: |
| | | | | Cataract, |
| | | | | autosomal |
| | | | | dominant |
| | | | | nuclear; |
| | | | | Cataract, |
| | | | | congenital |
| | | | | progressive, |
| | | | | autosomal |
| | | | | recessive; |
| | | | | Cataract, |
| | | | | zonular |
| | | | | central |
| | | | | nuclear, |
| | | | | autosomal |
| | | | | dominant. |
| HSPB1 | hsp27 | 1 × 10⁻⁰⁹ | Classic heat- | [OMIM: |
| | | | inducible | 602195]: |
| | | | sHsp. | Phenotypes of |
| | | | (Cardiac, | [HSP27]: |
| | | | skeletal, | Charcot- |
| | | | and smooth | Marie-Tooth |
| | | | muscles, | disease, |
| | | | brain, kidney, | axonal, |
| | | | testis, among | type 2F |
| | | | others in the | (cross-ref. |
| | | | absence of | OMIM: 606595); |
| | | | stress). | Neuropathy, |
| | | | | distal |
| | | | | hereditary |
| | | | | motor |
| | | | | (cross-ref. |
| | | | | OMIM: 608634). |
| HSPB2 | MKBP ^{‡} | 2 × 10⁻⁰⁸ | Striated | — |
| | | | muscles | |
| HSPB6 | hsp20 | 2 × 10⁻⁰⁷ | Cardiac, | — |
| | | | skeletal, | |
| | | | and smooth | |
| | | | muscles | |
| HSPB7 | cardio- | 5 × 10⁻⁰⁵ | Striated | — |
| | vascular | | muscles | |
| | hsp | | | |
| HSPB3 | hsp17 | 2 × 10⁻⁰⁴ | Striated | — |
| | | | muscles | |
| HSPB9 | — | 0.089 | Testis germ | Exclusive to |
| | | | cells | testis germ |
| | | | | cells and |
| | | | | only during |
| | | | | certain |
| | | | | stages of |
| | | | | spermato- |
| | | | | genesis |
| HSPB10 | ODFP ^{†} | 2.1 ^{††} | Sperm cell | ODFP occurs |
| | | | tails | exclusively |
| | | | | in the |
| | | | | axoneme of |
| | | | | sperm cells |

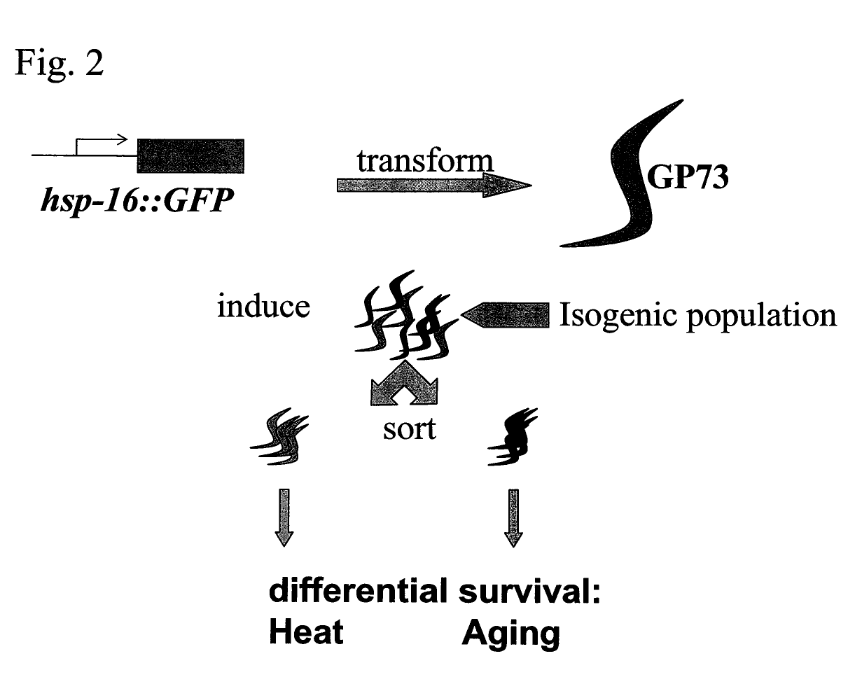

*BLASTP 2.2.14 [release May 07, 2006]. Database: All non-redundant GenBank CDS translations + PDB + SwissProt + PIR + PRF excluding environmental samples 3,794,442 sequences; 1,308,299,877 total letters (Jul. 19, 2006).
**See introduction of Fontaine et. al., Cell Stress & Chaperones (2003) 8 (1), 62-69 for references relating to individual sHSP distribution.
***OMIM (Online Mendelian Inheritance in Man).
^{‡}Myotonic Dystrophy Protein Kinase Binding Protein.
^{†}Sperm Outer Dense Fiber Protein.
^{††}ODFP is a bona fide member of the sHSP family in humans (Fontaine et. al., Cell Stress & Chaperones (2003) 8 (1), 62-69).

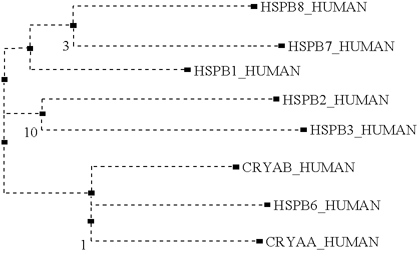 (n.b. For a tree containing HSPB9 and HSPB10 see FIG. 2 (Fontaine et. al., Cell Stress & Chaperones (2003) 8 (1), 62-69).)

In certain embodiments, analysis of the level of a longevity-predicting marker such as HSP42 in yeast or HSPB-like molecules in bacteria or virus may be assessed to determine the longevity or overall strength or health of an organism. The following references indicate comparable proteins exist in these species and likely reflect longevity-predicting markers in these organisms. Levels of these longevity-predicting markers may be used to assess the longevity of corresponding organism either from a sample or in a subject infected with the organism. (Madsen O, et a. (2004) Evolutionary diversity of vertebrate small heat shock proteins. J Mol Evol et. al. 59, 792-805; Haslbeck et. al. (2004) Hsp42 is the general small heat shock protein in the cytosol of Saccharomyces cerevisiae. EMBO J. 2004 Feb. 11; 23(3):638-49. Epub 2004 Jan. 29 Kappe, et. al. Evolution and diversity of prokaryotic small heat shock proteins. Prog. Mol. Subcell Biol. 28, 1-17 (2002). Laksanalamai P & Robb FT (2004) Small heat shock proteins from extremophiles: a review. Extremophiles 8, 1-11.; Sun et. al (2002) Small heat shock proteins and stress tolerance in plants. Biochim Biophys Acta 1577, 1-9; Waters, et. al E. Evolution, structure and function of the small heat shock proteins in plants. J. Exp. Bot 47, 325-338 (1996). hsp20 [Cyanophage P-SSM4] ACCESSION # AAX46942)

In certain embodiments, analysis of the level of a longevity-predicting marker such as HSP27 may be of use for therapeutic diagnosis and/or treatment of cancer. It is anticipated that any type of cancer and/or any type of tumor antigen may be targeted for diagnostic and/or therapeutic purposes. Exemplary types of tumors that may be targeted include, but are not limited to, acute lymphocytic leukemia, acute myelogenous leukemia, biliary cancer, breast cancer, cervical cancer, chronic lymphocytic leukemia, chronic myelogenous leukemia, colorectal cancer, endometrial cancer, esophageal cancer, gastric cancer, head and neck cancers, Hodgkin's lymphoma, lung cancer, medullary thyroid cancer, non-Hodgkin's lymphoma, multiple myeloma, renal cancer, ovarian cancer, pancreatic cancer, melanoma, liver cancer, prostate cancer, glial and other brain and spinal cord tumors, and urinary bladder cancer.

In other embodiments, analysis of the level of a longevity-predicting marker such as HSP27 may be of use to detect and/or treat infection by a pathogenic organisms, such as bacteria, viruses, fungi, unicellular parasites or macromolecules associated with a pathogenic organism. Exemplary fungi that may be treated include but are not limited to Cryptococcus neoformans, Histoplasma capsulatum, Blastomyces dermatitidis, Candida albican or combination thereof. Exemplary viruses include but are not limited to human immunodeficiency virus (HIV), herpes virus, cytomegalovirus, influenza virus, human papilloma virus, hepatitis B virus, hepatitis C virus, Sendai virus, feline leukemia virus, Reo virus, polio virus, human serum parvo-like virus, simian virus 40, respiratory syncytial virus, Varicella-Zoster virus, Dengue virus, rubella virus, measles virus, adenovirus, human T-cell leukemia viruses, Epstein-Barr virus, murine leukemia virus, mumps virus, vesicular stomatitis virus, or combination thereof. Exemplary bacteria include but are not limited to Streptococcus agalactiae, Legionella pneumophilia, Streptococcus pyogenes, Escherichia coli, Neisseria gonorrhoeae, Neisseria meningitidis, Pneumococcus spp., Mycobacterium leprae, Brucella abortus, Pseudomonas aeruginosa, Mycobacterium tuberculosis, Mycoplasma pneumonia or combination thereof. Exemplary parasites include but are not limited to Giardia lamblia, Giardia spp., Toxoplasma gondii, Cryptospordium spp., Leishmania spp., Trypanosoma evansi, Dientamoeba fragilis, Trichomonas vaginalis, Plasmodium falciparum, Isospora spp., Toxoplasma spp. Enterocytozoon spp., Pneumocystis spp., Balantidium spp or combination thereof.

Marker Administration to a Sample

Various embodiments of the claimed methods and/or compositions may concern one or more longevity-predicting marker complex to be administered to a sample obtained from a subject. It is contemplated herein that a longevity-predicting marker complex such as an alpha B crystallin-like HSP27 or a derivative of a HSP27-like molecule linked to a reporter molecule (e.g. green fluorescent protein) can be generated by means known in the art. These complexes may be introduced to any sample or organism disclosed herein and the level of these constructs assessed. In accordance with these embodiments, the longevity or lifespan of a sample or organism can be assessed. In addition, these in vitro samples may be tested for affects of an agent on the sample by assessing the level of longevity-predicting marker(s) before and after treatment with an agent.

Marker Assessment in a Subject

Various embodiments of the claimed methods and/or compositions may concern measuring the levels of one or more longevity-predicting marker in a sample from a subject. Samples from a subject may include, but are not limited to, urine, blood, tissue, saliva, vaginal, dermal, amniotic fluid, cerebrospinal fluid, tears or combination thereof

In other embodiments, it is contemplated that a longevity-predicting marker complexed to a reporter gene may be introduced to a target tissue of a subject to assess the expression of the complex in the target tissue. In accordance with these embodiments, the level of expression of the complex is indicative of the health of the target tissue, a high level of expression of the complex is indicative of a healthy target tissue and a low level of expression of the complex is indicative of the presence of an unhealthy condition. If low levels of the longevity-predicting marker are expressed, a treating healthcare provider can administer additional tests and/or administer a therapeutic treatment to the subject. In addition, the subject can also be more closely monitored for the onset of a condition of the target tissue by periodically assessing the level of expression of a complex administered to the target tissue of the subject.

Other Aging Assessment Tests

In one exemplary method, the assessment of levels of the longevity-predicting markers such as HSP27 disclosed in the present invention may be combined with any age assessment tests. For example, using microarray data sets or analysis in combination with the assessment tests of the present invention. In one particular embodiment, gene expression is known to become more variable with advancing age. This age-correlated heterogeneity of expression (ACHE) has a minor effect on individual genes, but is widespread throughout the transcriptome. ACHE is another outcome of the accumulation of stochastic effects at the cellular level. A statistical test that measures an increase in expression heterogeneity with age. ACHE is calculated separately for a given probe set (a probe set detects one mRNA transcript in microarray experiments). Any known age assessment test such as a microarray analysis of particular set or sets of genes may be used in combination with measuring the levels of a longevity-predicting marker molecule(s).

Peptide Preparation

Methods for chemically modifying peptides to render them less susceptible to degradation by endogenous proteases or more absorbable are well known (see, for example, Blondelle et al., 1995, Biophys. J. 69:604-11; Ecker and Crooke, 1995, Biotechnology 13:351-69). Methods for preparing libraries of peptide analogs, such as peptides containing D-amino acids; peptidomimetics consisting of organic molecules that mimic the structure of a peptide; or peptoids such as vinylogous peptoids, have also been described and may be used to construct peptide based bioactive assemblies suitable for oral administration to a subject. Peptide stabilization may also occur by substitution of D-amino acids for naturally occurring L-amino acids, particularly at locations where endopeptidases are known to act.

In certain embodiments, a standard peptide bond linkage may be replaced by one or more alternative linking groups, such as CH₂—NH, CH₂—S, CH₂—CH₂, CH═CH, CO—CH₂, CHOH—CH₂ and the like. Methods for preparing peptide mimetics are well known in the art. (for example Holladay et al., 1983, Tetrahedron Lett. 24:4401-04; and Almquiest et al., 1980, J. Med. Chem. 23:1392-98). Peptide mimetics may exhibit enhanced stability and/or absorption in vivo compared to their peptide analogs.

In one embodiment, a longevity-predicting marker complex may be linked to a reporter molecule by designing a plasmid or any other known expression molecule capable of producing a longevity marker-complex, such as a nucleic acid sequence.

In still other embodiments, peptides may be modified for oral or inhalational administration by conjugation to certain proteins.

It is contemplated that any longevity-predicting marker complex disclosed herein may be delivered encapsulated by methods known in the art such as within a gel, a microbead, a microparticle, a matrix formulation or the like. It is also contemplated that any longevity-marker complex disclosed herein and administered to a subject or a sample may be administered as a rapid release formulation or a time released formulation.

Nucleic Acids

As described herein, an aspect of the present disclosure concerns isolated and/or synthetically derived nucleic acids and methods of use of isolated nucleic acids (e.g. detection molecules). In certain embodiments, the nucleic acid sequences disclosed herein have utility as hybridization probes or amplification primers. These nucleic acids may be used, for example, in diagnostic evaluation of tissue samples. In certain embodiments, these probes and primers consist of oligonucleotide fragments. Such fragments should be of sufficient length to provide specific hybridization to a RNA or DNA tissue sample. The sequences typically will be 10-40 nucleotides, but may be longer. Longer sequences, e.g., 45, 50, 100, 500 and even up to full length, are preferred for certain embodiments.

Nucleic acid molecules having contiguous stretches of about 1, 5, 10, 20, 30, 40, 50, 60, 70, 80, 90, 100, 125, 150, 175, 200, 250, 300, 400, 500, 600, 750, 1000, 1500, 2000, 2500 or more nucleotides from a sequence selected from the disclosed nucleic acid sequences are contemplated. Molecules that are complementary to the above mentioned sequences and that bind to these sequences under high stringency conditions also are contemplated. These probes will be useful in a variety of hybridization embodiments, such as Southern and Northern blotting.

The use of a hybridization probe of between 14 and 100 nucleotides in length allows the formation of a duplex molecule that is both stable and selective. Molecules having complementary sequences over stretches greater than 20 bases in length are generally preferred, in order to increase stability and selectivity of the hybrid, and thereby improve the quality and degree of particular hybrid molecules obtained. One will generally prefer to design nucleic acid molecules having stretches of 20 to 30 nucleotides, or even longer where desired. Such fragments may be readily prepared by, for example, directly synthesizing the fragment by chemical means or by introducing selected sequences into recombinant vectors for recombinant production.

Accordingly, the nucleotide sequences of the invention may be used for their ability to selectively form duplex molecules with complementary stretches of genes or RNAs or to provide primers for amplification of DNA or RNA from tissues. Depending on the application envisioned, one may desire to employ varying conditions of hybridization to achieve varying degrees of selectivity of probe towards target sequence.

For applications requiring high selectivity, one will typically desire to employ relatively stringent conditions to form the hybrids, e.g., one will select relatively low salt and/or high temperature conditions, such as provided by about 0.02 M to about 0.10 M NaCl at temperatures of about 50° C. to about 70° C. Such high stringency conditions tolerate little, if any, mismatch between the probe and the template or target strand, and would be particularly suitable for isolating specific genes or detecting specific mRNA transcripts. It is generally appreciated that conditions can be rendered more stringent by the addition of increasing amounts of formamide.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. or other known conditions in the art.

In certain embodiments, it will be advantageous to employ nucleic acid sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, calorimetric indicator substrates are known which can be employed to provide a detection means visible to the human eye or spectrophotometrically, to identify specific hybridization with complementary nucleic acid-containing samples.

In general, it is envisioned that the hybridization probes described herein will be useful both as reagents in solution hybridization, as in PCR, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The selected conditions will depend on the particular circumstances based on the particular criteria required (depending, for example, on the G+C content, type of target nucleic acid, source of nucleic acid, size of hybridization probe, etc.). Following washing of the hybridized surface to remove non-specifically bound probe molecules, hybridization is detected, or even quantified, by means of the label.

It will be understood that the present invention is not limited to the particular probes disclosed herein and particularly is intended to encompass at least nucleic acid sequences that are hybridizable to the disclosed sequences or are functional sequence analogs of these sequences. For example, a partial sequence may be used to identify a structurally-related gene or the full length genomic or cDNA clone from which it is derived. Those of skill in the art are well aware of the methods for generating cDNA and genomic libraries which can be used as a target for the above-described probes (Sambrook et al., 1989).

For applications in which the nucleic acid segments of the present invention are incorporated into vectors, such as plasmids, cosmids or viruses, these segments may be combined with other DNA sequences, such as promoters, polyadenylation signals, restriction enzyme sites, multiple cloning sites, other coding segments, and the like, such that their overall length may vary considerably. It is contemplated that a nucleic acid fragment of almost any length may be employed, with the total length preferably being limited by the ease of preparation and use in the intended recombinant DNA protocol.

DNA segments encoding a specific gene may be introduced into recombinant host cells and employed for expressing a specific part of or entire structural or regulatory protein. Alternatively, through the application of genetic engineering techniques, subportions or derivatives of selected genes may be employed. Upstream regions containing regulatory regions such as promoter regions may be isolated and subsequently employed for expression of the selected gene.

Where an expression product is to be generated, it is possible for the nucleic acid sequence to be varied while retaining the ability to encode the same product. Reference to a codon chart will permit those of skill in the art to design any nucleic acid encoding for the product of a given nucleic acid.

Nucleic Acid Delivery

Liposomal Formulations

In certain broad embodiments of the invention, the oligo- or polynucleotides and/or expression vectors may be entrapped in a liposome for delivery to a subject or sample. Also, contemplated herein are cationic lipid-nucleic acid complexes, such as lipofectamine-nucleic acid complexes.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al, 1991). In yet further embodiments, the liposome may be complexed or employed in conjunction with both HVJ and HMG-1. In that such expression vectors have been successfully employed in transfer and expression of a polynucleotide in vitro and in vivo, then they are applicable for the present invention. Where a bacterial promoter is employed in the DNA construct, it also will be desirable to include within the liposome an appropriate bacterial polymerase.

Lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform, chloroform/methanol or t-butanol can be stored at about −20° C. Preferably, chloroform is used as the only solvent since it is more readily evaporated than methanol.

Liposomes used according to the present invention can be made by different methods known in the art. The size of the liposomes varies depending on the method of synthesis.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum. In the alternative, liposomes can be prepared in accordance with any known procedure in the art.

The dried lipids or lyophilized liposomes prepared as described above may be reconstituted in a solution of nucleic acid and diluted to an appropriate concentration with a suitable solvent, e.g., DPBS. After isolating only the complexed liposome and determination of the amount of nucleic acid encapsulated in the liposome preparation, the liposomes may be diluted to appropriate concentration and stored at 4° C. until use.

Alternative Delivery Systems

Viral vectors may be employed as expression constructs in the present invention. Vectors derived from viruses such as vaccinia virus, and herpes viruses may be employed. They offer several attractive features for various mammalian cells.

Several non-viral methods for the transfer of expression vectors into cultured mammalian cells also are contemplated by the present invention. These include, but are not limited to, calcium phosphate precipitation (Graham and Van Der Eb, 1973; Chen and Okayama, 1987; Rippe et al., 1990) DEAE-dextran (Gopal, 1985), lipofectamine-DNA complexes, and receptor-mediated transfection (Wu and Wu, 1987; Wu and Wu, 1988). Some of these techniques may be successfully adapted for in vivo or ex vivo use.

In one embodiment of the invention, the expression construct may simply consist of naked recombinant vector. Transfer of the construct may be performed by any of the methods mentioned above which physically or chemically permeabilize the cell membrane. For example, Dubensky et al. (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes.

Expressed Proteins

It is contemplated herein that a gene of the present invention can be inserted into an appropriate expression system. The gene can be expressed in any number of different recombinant DNA expression systems to generate large amounts of the polypeptide product, which can then be purified and used to vaccinate animals to generate antisera to particular sites of the protein with which further studies may be conducted.

Examples of expression systems known to the skilled practitioner in the art include bacteria such as E. coli, yeast such as Pichia pastoris, baculovirus, and mammalian expression systems such as in Cos or CHO cells. A complete gene can be expressed or, alternatively, fragments of the gene encoding portions of polypeptide can be produced.

Computer sequence analysis may be used to determine the location of the predicted major antigenic determinant epitopes of any longevity-predicting marker polypeptide disclosed herein. Software capable of carrying out this analysis is readily available commercially, for example MacVector (IBI, New Haven, Conn.). The software typically uses standard algorithms such as the Kyte/Doolittle or Hopp/Woods methods for locating hydrophilic sequences may be found on the surface of proteins and are, therefore, likely to act as antigenic determinants.

Once this analysis is made, polypeptides may be prepared which contain at least the essential features of the antigenic determinant and which may be employed in the generation of antisera against the polypeptide. Minigenes or gene fusions encoding these determinants may be constructed and inserted into expression vectors by standard methods, for example, using PCR cloning methodology. Some of these systems produce recombinant polypeptides bearing only a small number of additional amino acids, which are unlikely to affect the antigenic ability of the recombinant polypeptide.

As an alternative to recombinant polypeptides, synthetic peptides corresponding to the antigenic determinants may be prepared. Such peptides are at least six amino acid residues long, and may contain up to approximately 35 residues, which is the approximate upper length limit of automated peptide synthesis machines, such as those available from Applied Biosystems (Foster City, Calif.). Use of such small peptides for vaccination typically requires conjugation of the peptide to an immunogenic carrier protein such as hepatitis B surface antigen, keyhole limpet hemocyanin or bovine serum albumin. Methods for performing this conjugation are well known in the art.

In the alternative, known antibodies may be used to detect the level of longevity-predicting markers in a sample or a subject. Alpha-B crystallin, alpha-A crystallin and alpha-B cystallin-like (e.g. HSP27) directed antibodies are available at least at Abcam Inc. or Upstate (Cambridge, Mass.; Lake Placid, NY, respectively).

Another method for the preparation of the polypeptides according to the invention is the use of peptide mimetics. Mimetics are peptide-containing molecules which mimic elements of protein secondary structure. See, for example, Johnson et al., “Peptide Turn Mimetics” in BIOTECHNOLOGY AND PHARMACY, Pezzuto et al., Eds., Chapman and Hall, New York (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule.

Successful applications of the peptide mimetic concept have thus far focused on mimetics of β-turns within proteins, which are known to be highly antigenic. Likely β-turn structure within a polypeptide may be predicted by computer-based algorithms as discussed herein. Once the component amino acids of the turn are determined, peptide mimetics may be constructed to achieve a similar spatial orientation of the essential elements of the amino acid side chains.

The engineering of DNA segment(s) for expression in a prokaryotic or eukaryotic system may be performed by techniques generally known to those of skill in recombinant expression. It is believed that virtually any expression system may be employed in the expression of the claimed nucleic acid sequences.

To express a recombinant encoded protein or peptide, whether mutant or wild-type, in accordance with the present invention one could prepare an expression vector that comprises one of the claimed isolated nucleic acids under the control of, or operatively linked to, one or more promoters. To bring a coding sequence “under the control of” a promoter, one positions the 5′ end of the transcription initiation site of the transcriptional reading frame generally between about 1 and about 50 nucleotides “downstream” (i.e., 3′) of the chosen promoter. The “upstream” promoter stimulates transcription of the DNA and promotes expression of the encoded recombinant protein.

Many standard techniques are available to construct expression vectors containing the appropriate nucleic acids and transcriptional/translational control sequences in order to achieve protein or peptide expression in a variety of host-expression systems. Cell types available for expression include, but are not limited to, bacteria, such as E. coli and B. subtilis transformed with recombinant bacteriophage DNA, plasmid DNA or cosmid DNA expression vectors. In general, plasmid vectors containing replicon and control sequences which are derived from species compatible with the host cell are used in connection with these hosts.

Examples of useful mammalian host cell lines are VERO and HeLa cells, Chinese hamster ovary (CHO) cell lines, W138, BHK, COS-7, 293, HepG2, 3T3, RIN and MDCK cell lines. In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the encoded protein.

Different host cells have characteristic and specific mechanisms for the post-translational processing and modification of proteins. Appropriate cells lines or host systems may be chosen to ensure the correct modification and processing of the foreign protein expressed. Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences.

Specific initiation signals may also be required for efficient translation of the claimed isolated nucleic acid coding sequences. These signals include the ATG initiation codon and adjacent sequences. Exogenous translational control signals, including the ATG initiation codon, may additionally need to be provided. One of ordinary skill in the art would readily be capable of determining this and providing the necessary signals. It is well known that the initiation codon must be in-frame (or in-phase) with the reading frame of the desired coding sequence to ensure translation of the entire insert. These exogenous translational control signals and initiation codons may be of a variety of origins, both natural and synthetic. The efficiency of expression may be enhanced by the inclusion of appropriate transcription enhancer elements or transcription terminators (Bittner et al, 1987).

It is contemplated that the isolated nucleic acids of the invention may be “overexpressed”, i.e., expressed in increased levels relative to its natural expression in human prostate, bladder or breast cells, or even relative to the expression of other proteins in the recombinant host cell. Such overexpression may be assessed by a variety of methods, including radio-labeling and/or protein purification. However, simple and direct methods are preferred, for example, those involving SDS/PAGE and protein staining or Western blotting, followed by quantitative analyses, such as densitometric scanning of the resultant gel or blot. A specific increase in the level of the recombinant protein or peptide in comparison to the level in natural human prostate, bladder or breast cells is indicative of overexpression, as is a relative abundance of the specific protein in relation to the other proteins produced by the host cell and, e.g., visible on a gel.

Aptamers

In certain embodiments, a longevity-predicting marker disclosed herein may be detected by an aptamer. Methods of constructing and determining the binding characteristics of aptamers are well known in the art. For example, such techniques are described in U.S. Pat. Nos. 5,582,981, 5,595,877 and 5,637,459, each incorporated herein by reference. Aptamers may be prepared by any known method, including synthetic, recombinant, and purification methods, and may be used alone or in combination with other ligands specific for the same target. In general, a minimum of approximately 3 nucleotides, preferably at least 5 nucleotides, are necessary to effect specific binding. Aptamers of sequences shorter than 10 bases may be feasible, although aptamers of 10, 20, 30 or 40 nucleotides may be preferred.

Aptamers need to contain the sequence that confers binding specificity, but may be extended with flanking regions and otherwise derivatized. In preferred embodiments, alpha B crystalline-like molecule recognizing region of aptamers may be flanked by primer-binding sequences, facilitating the amplification of the aptamers by PCR or other amplification techniques known in the art. In a further embodiment, the flanking sequence may comprise a specific sequence that preferentially recognizes or binds a moiety to enhance the immobilization of the aptamer to a substrate.

Aptamers may be isolated, sequenced, and/or amplified or synthesized as conventional DNA or RNA molecules. Alternatively, aptamers of interest may comprise modified oligomers. Any of the hydroxyl groups ordinarily present in aptamers may be replaced by phosphonate groups, phosphate groups, protected by a standard protecting group, or activated to prepare additional linkages to other nucleotides, or may be conjugated to solid supports. One or more phosphodiester linkages may be replaced by alternative linking groups, such as P(O)O replaced by P(O)S, P(O)NR₂, P(O)R, P(O)OR′, CO, or CNR₂, wherein R is H or alkyl (1-20C) and R′ is alkyl (1-20C); in addition, this group may be attached to adjacent nucleotides through O or S. Not all linkages in an oligomer need to be identical.

The aptamers used as starting materials in the process of the invention to determine specific binding sequences may be single-stranded or double-stranded DNA or RNA. In a preferred embodiment, the sequences are single-stranded DNA, which is less susceptible to nuclease degradation than RNA. In preferred embodiments, the starting aptamer will contain a randomized sequence portion, generally including from about 10 to 400 nucleotides, more preferably 20 to 100 nucleotides. The randomized sequence is flanked by primer sequences that permit the amplification of aptamers found to bind to the target. For synthesis of the randomized regions, mixtures of nucleotides at the positions where randomization is desired may be added during synthesis.

Methods for preparation and screening of aptamers that bind to particular targets of interest are well known, for example U.S. Pat. No. 5,475,096 and U.S. Pat. No. 5,270,163, each incorporated by reference. The technique generally involves selection from a mixture of candidate aptamers and step-wise iterations of binding, separation of bound from unbound aptamers and amplification. Because only a small number of sequences (possibly only one molecule of aptamer) corresponding to the highest affinity aptamers exist in the mixture, it is generally desirable to set the partitioning criteria so that a significant amount of aptamers in the mixture (approximately 5-50%) are retained during separation. Each cycle results in an enrichment of aptamers with high affinity for the target. Repetition for between three to six selection and amplification cycles may be used to generate aptamers that bind with high affinity and specificity to the target, such as a longevity-predicting marker such as HSP27 or other alpha-B crystallin-like molecules.

Proteins and Peptides

A variety of polypeptides or proteins may be used within the scope of the claimed methods and compositions. In certain embodiments, the proteins can include proteins such as alpha-B crystallin proteins, alpha-B crystallin-like proteins or antibodies or fragments of antibodies containing an antigen-binding site to a alpha-B crystallin protein. As used herein, a protein, polypeptide or peptide generally refers, but is not limited to, a protein of greater than about 200 amino acids, up to a full length sequence translated from a gene; a polypeptide of greater than about 100 amino acids; and/or a peptide of from about 3 to about 100 amino acids. For convenience, the terms “protein,” “polypeptide” and “peptide” are used interchangeably herein. Accordingly, the term “protein or peptide” encompasses amino acid sequences comprising at least one of the 20 common amino acids found in naturally occurring proteins, or at least one modified or unusual amino acid.

As used herein, an “amino acid residue” refers to any naturally occurring amino acid, any amino acid derivative or any amino acid mimic known in the art. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties.

Accordingly, the term “protein or peptide” encompasses amino acid sequences comprising at least one of the 20 common amino acids found in naturally occurring proteins, or at least one modified or unusual amino acid known in the art. Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed and may be found at computerized databases known to those of ordinary skill in the art. One such database is the National Center for Biotechnology Information's Genbank and GenPept databases (www.ncbi.nlm.nih.gov/). The coding regions for known genes may be amplified and/or expressed using the techniques disclosed herein or as would be know to those of ordinary skill in the art. Alternatively, various commercial preparations of proteins, polypeptides, and peptides are known to those of skill in the art.

Fusion Proteins

Various embodiments may concern fusion proteins. These molecules generally have all or a substantial portion of a peptide, linked at the N- or C-terminus, to all or a portion of a second polypeptide or protein. Methods of generating fusion proteins are well known to those of skill in the art. Such proteins may be produced, for example, by chemical attachment using bifunctional cross-linking reagents, by de novo synthesis of the complete fusion protein, or by attachment of a DNA sequence encoding a first protein or peptide to a DNA sequence encoding a second peptide or protein, followed by expression of the intact fusion protein.

Synthetic Peptides

Proteins or peptides may be synthesized, in whole or in part, in solution or on a solid support in accordance with conventional techniques. Various automatic synthesizers are commercially available and can be used in accordance with known protocols. See, for example, Stewart and Young, (1984, Solid Phase Peptide Synthesis, 2d. ed., Pierce Chemical Co.); Tam et al., (1983, J. Am. Chem. Soc., 105:6442); Merrifield, (1986, Science, 232: 341-347); and Barany and Merrifield (1979, The Peptides, Gross and Meienhofer, eds., Academic Press, New York, pp. 1-284). Short peptide sequences, usually from about 6 up to about 35 to 50 amino acids, can be readily synthesized by such methods. Alternatively, recombinant DNA technology may be employed wherein a nucleotide sequence which encodes a peptide of interest is inserted into an expression vector, transformed or transfected into an appropriate host cell, and cultivated under conditions suitable for expression.

Antibodies

Various embodiments may concern antibodies for a target. The term “antibody” is used herein to refer to any antibody-like molecule that has an antigen binding region, and includes antibody fragments such as Fab′, Fab, F(ab′)₂, single domain antibodies (DABs), Fv, scFv (single chain Fv), and the like. Techniques for preparing and using various antibody-based constructs and fragments are well known in the art. Means for preparing and characterizing antibodies are also well known in the art (See, e.g., Harlowe and Lane, 1988, Antibodies: A Laboratory Manual, Cold Spring Harbor Laboratory). Antibodies of use may also be commercially obtained from a wide variety of known sources. For example, a variety of antibody secreting hybridoma lines are available from the American Type Culture Collection (ATCC, Manassas, Va.). A large number of antibodies against various disease targets, including but not limited to tumor-associated antigens, have been deposited at the ATCC and are available for use in the claimed methods and compositions. (See, for example, U.S. Pat. Nos. 7,060,802; 7,056,509; 7,049,060).

Production of Antibody Fragments

Some embodiments of the claimed methods and/or compositions may concern antibody fragments. Such antibody fragments may be obtained by pepsin or papain digestion of whole antibodies by conventional methods. For example, antibody fragments may be produced by enzymatic cleavage of antibodies with pepsin to provide F(ab′)₂ fragments. This fragment may be further cleaved using a thiol reducing agent and, optionally, followed by a blocking group for the sulfhydryl groups resulting from cleavage of disulfide linkages, to produce Fab′ monovalent fragments. Alternatively, an enzymatic cleavage using for example, papain produces two monovalent Fab fragments and an Fc fragment. Exemplary methods for producing antibody fragments are disclosed in U.S. Pat. No. 4,036,945; and U.S. Pat. No. 4,331,647).

It is contemplated herein that any antibody or antibody fragment used to detect the presence or level of a longevity predicting marker complex may be a chimeric, human or humanized antibody generated by means known in the art. Known antibodies to bind the longevity marker complex include but are not limited to anti-HSP-27 antibody, anti-HSP-27-like antibody.

Methods of Disease Tissue Detection, Diagnosis and Imaging

Protein-Based In Vitro Diagnosis

The present invention contemplates the use of longevity-predicting markers to assess conditions of a biological sample in vitro and/or in vivo for the expression of the marker (e.g. HSP27). In exemplary assays, a marker can be detected or measured by associating with a macromolecule such as a protein, peptide, nucleic acid, an antibody, fusion protein, or fragment thereof by a liquid phase or bound to a solid-phase carrier, as described below. The skilled artisan will realize that a wide variety of techniques are known for determining levels of expression of a particular gene and any such known method, such as immunoassay, RT-PCR, mRNA purification and/or cDNA preparation followed by hybridization to a gene expression assay chip may be utilized to determine levels of expression in individual subjects and/or tissues. Exemplary in vitro assays of use include RIA, ELISA, sandwich ELISA, Western blot, slot blot, dot blot, and the like. Although such techniques were developed using intact antibodies, bioactive assemblies that incorporate antibodies, antibody fragments or other binding moieties may be used.

Longevity-predicting markers can be additionally labeled with any other appropriate marker moiety, for example, a radioisotope, an enzyme, a fluorescent label, a dye, a chromogen, a chemiluminescent label, a bioluminescent label or a paramagnetic label. The marker moiety may be a radioisotope that is detected by such means as the use of a gamma counter or a beta-scintillation counter or by autoradiography.

Immunohistochemistry

The antibodies of the present invention may be used in conjunction with both fresh-frozen and formalin-fixed, paraffin-embedded tissue blocks prepared by immunohistochemistry (IHC). Any IHC method well known in the art may be used such as those described in Diagnostic Immunopathology, 2nd edition. edited by, Robert B. Colvin, Atul K. Bhan and Robert T. McCluskey. Raven Press, New York., 1995, (incorporated herein by reference) and in particular, Chapter 31 of that reference entitled Gynecological and Genitourinary Tumors (pages 579-597), by Debra A. Bell, Robert H. Young and Robert E. Scully and references therein.

ELISA

As noted, it is contemplated that the encoded proteins or peptides or other molecules of the present invention may be detected, e.g., in immunohistochemistry assays and in ELISA assays. One evident utility of these molecules is in immunoassays for the detection of longevity predicting marker proteins, as needed in diagnosis and prognostic monitoring.

Immunoassays, such as enzyme linked immunosorbent assays (ELISAs) and radioimmunoassays (RIA) are known in the art. Immunohistochemical detection using tissue sections is also particularly useful. However, it will be readily appreciated that detection is not limited to such techniques, and Western blotting, dot blotting, FACS analyses, gold-labelled secondary antibodies, and the like may also be used.

In one exemplary ELISA, antibodies binding to an encoded protein of the invention are immobilized onto a selected surface exhibiting protein affinity, such as a well in a polystyrene microtiter plate. Then, a test composition, such as a clinical sample, is added to the wells. After binding and washing to remove non-specifically bound immunecomplexes, the bound antigen may be detected. Detection is generally achieved by the addition of a second antibody specific for the target protein, that is linked to a detectable label. This type of ELISA is a simple “sandwich ELISA”. Detection may also be achieved by the addition of a second antibody, followed by the addition of a third antibody that has binding affinity for the second antibody, with the third antibody being linked to a detectable label.

In another exemplary ELISA, the samples suspected of containing the longevity predicting marker antigen are immobilized onto the well surface and then contacted with the antibodies of the invention. After binding and washing to remove non-specifically bound immunecomplexes, the bound antigen is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Another ELISA in which the proteins or peptides are immobilized, involves the use of antibody competition in the detection. In this ELISA, labeled antibodies are added to the wells, allowed to bind to the marker protein, and detected by means of their label. The amount of marker antigen in an unknown sample is then determined by mixing the sample with the labeled antibodies before or during incubation with coated wells. The presence of marker antigen in the sample acts to reduce the amount of antibody available for binding to the well and thus reduces the ultimate signal. This is appropriate for detecting antibodies in an unknown sample, where the unlabeled antibodies bind to the antigen-coated wells and also reduces the amount of antigen available to bind the labeled antibodies.

It is contemplated herein that any known peptide, protein or nucleic acid detection system may be used to detect the presence of or level of a longevity-predicting marker as disclosed in the present invention.

To provide a detecting means, a second or third antibody can be associated with a detectible label. In one example, this will be an enzyme that will generate color development upon incubating with an appropriate chromogenic substrate. Thus, for example, one will desire to contact and incubate the first or second immunecomplex with a urease, glucose oxidase, alkaline phosphatase or hydrogen peroxidase-conjugated antibody for a period of time and under conditions that favor the development of further immunecomplex formation (e.g., incubation for 2 hours at room temperature in a PBS-containing solution such as PBS-Tween). Quantitation can be achieved in this example by measuring the degree of color generation, e.g., using a visible spectra spectrophotometer.

In Vivo Diagnosis

Methods of diagnostic imaging with labeled macromolecules are well-known. For example, in the technique of immunoscintigraphy, ligands or antibodies are labeled with a gamma-emitting radioisotope and introduced into a patient. A gamma camera is used to detect the location and distribution of gamma-emitting radioisotopes.

The radiation dose delivered to the patient is maintained at as low a level as possible through the choice of isotope for the best combination for a primary or secondary detection of a complex (radiolabelled marker, may be the primary agent detected) of minimum half-life, minimum retention in the body, and minimum quantity of isotope which will permit detection and accurate measurement.

Imaging Agents for Secondary Detection of a Longevity-Predicting Marker Complex and Radioisotopes

Many appropriate imaging agents are known in the art, as are methods for their attachment to proteins or peptides (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the protein or peptide (U.S. Pat. No. 4,472,509). Proteins or peptides also may be reacted with an enzyme in the presence of a coupling agent such as glutaraldehyde or periodate. Conjugates with fluorescein markers are prepared in the presence of these coupling agents or by reaction with an isothiocyanate.

Non-limiting examples of paramagnetic ions of potential use as imaging agents include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III).

Radioisotopes of potential use as imaging or therapeutic agents include astatine²¹¹, carbon⁴, chromium⁵¹, chlorine³⁶, cobalt⁵⁷, cobalt⁵⁸, copper⁶², copper⁶⁴, copper⁶⁷, Eu¹⁵², fluorine¹⁸, gallium⁶⁷, gallium⁶⁸, hydrogen³, iodine¹²³, iodine¹²⁴, iodine¹²⁵, iodine¹³¹, indium¹¹¹, iron⁵², iron⁵⁹, lutetium¹⁷⁷, phosphorus32, phosphorus³³, rhenium¹⁸⁶, rhenium¹⁸⁸, Sc⁴⁷, selenium⁷⁵, silver¹¹¹, sulphur³⁵, technetium^{94m}, technetium^{99m}, yttrium⁸⁶ and yttrium⁹⁰, and zirconium⁸⁹. I¹²⁵ is often being preferred for use in certain embodiments, and technetium^{99m} and indiuml¹¹¹ are also often preferred due to their low energy and suitability for long-range detection.

Radioactively labeled proteins or peptides may be produced according to well-known methods in the art. Intermediary functional groups which are often used to bind radioisotopes which exist as metallic ions to peptides include diethylenetriaminepentaacetic acid (DTPA), DOTA, NOTA, porphyrin chelators and ethylene diaminetetracetic acid (EDTA). Also contemplated for use are fluorescent labels, including rhodamine, fluorescein isothiocyanate and renographin.

In certain embodiments, the proteins or peptides may be linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. In alternative embodiments, macromolecules of a Be complex may be tagged with a fluorescent marker.

In various embodiments, labels of use may comprise alternative metal nanoparticles. Methods of preparing nanoparticles are known. (See e.g., U.S. Pat. Nos. 6,054,495; 6,127,120; 6,149,868; Lee and Meisel, J. Phys. Chem. 86:3391-3395, 1982.) Nanoparticles may also be obtained from commercial sources (e.g., Nanoprobes Inc., Yaphank, NY; Polysciences, Inc., Warrington, Pa.). Modified nanoparticles are available commercially, such as Nanogold® nanoparticles from Nanoprobes, Inc. (Yaphank, NY). Functionalized nanoparticles of use for conjugation to proteins or peptides may be commercially obtained.

Chemotherapeutic Agents

In certain embodiments, chemotherapeutic agents may be administered once the level of longevity-predicting marker(s) of a subject or sample is determined. Anti-cancer chemotherapeutic agents of use include, but are not limited to, 5-fluorouracil, bleomycin, busulfan, camptothecins, carboplatin, chlorambucil, cisplatin (CDDP), cyclophosphamide, dactinomycin, daunorubicin, doxorubicin, estrogen receptor binding agents or combination thereof

Chemotherapeutic agents and methods of administration, dosages, etc., are well known to those of skill in the art (see for example, the “Physicians Desk Reference”, Goodman & Gilman's “The Pharmacological Basis of Therapeutics” and in “Remington's Pharmaceutical Sciences”, incorporated herein by reference in relevant parts). Some variation in dosage will necessarily occur depending on the condition of the subject being treated. The person responsible for administration will, in any event, determine the appropriate dose for the individual subject.

Pharmaceutical Compositions

In one embodiment, it is contemplated that a low level of longevity-predicting marker in a sample or a subject may require further tests to uncover a condition present in a subject. It is contemplated herein that the subject may then require therapeutic intervention. In accordance with this embodiment, a therapeutic agent may include, but is not limited to one or more of a drug, a toxin, a prodrug, a toxin, an enzyme, a protease, an enzyme-inhibitor, a nuclease, a hormone, a hormone antagonist, an anti-inflammatory agent, an anti-cancer agent, an immunomodulator, an oligonucleotide, a boron compound, a photoactive agent or combinations thereof

Other therapeutic agents contemplated for use herein may include but are not limited to one or more of the following: azacytidine, bleomycin, busulfan, camptothecin, carmustine, celebrex, chlorambucil, cisplatin, irinotecan (CPT-11), SN-38, carboplatin, cladribine, cyclophosphamide, cytarabine, daunomycin glucuronide, daunorubicin, dexamethasone, diethylstilbestrol, doxorubicin, epirubicin glucuronide, estramustine, etoposide, etoposide glucuronide, etoposide phosphate, floxuridine (FUdR), 3′,5′-O-dioleoyl-FudR (FUdR-dO), fludarabine, flutamide, fluorouracil, gemcitabine, hydroxyprogesterone caproate, hydroxyurea, idarubicin, L-asparaginase, leucovorin, lomustine, mechlorethamine, medroprogesterone acetate, megestrol acetate, melphalan, mercaptopurine, 6-mercaptopurine, methotrexate, mitomycin, mitotane, phenyl butyrate, prednisone, paclitaxel, pentostatin, tamoxifen, taxanes, taxol, testosterone propionate, thalidomide, thioguanine, thiotepa, teniposide, topotecan, uracil mustard, vinblastine, vinorelbine, vincristine, ricin, abrin, ribonuclease, onconase, rapLRl, DNase I, Staphylococcal enterotoxin-A, pokeweed antiviral protein, gelonin, diphtheria toxin, Pseudomonas exotoxin, Pseudomonas endotoxin, nitrogen mustard, ethyleneimine derivative, alkyl sulfonate, nitrosourea, triazene, folic acid analog, anthracycline, COX-2 inhibitor, pyrimidine analog, purine analog, antibiotic, epipodophyllotoxin, platinum coordination complex, vinca alkaloid, substituted urea, methyl hydrazine derivative, adrenocortical suppressant, antagonist, endostatin, cytokine, interleukin, interferon, lymphokine, tumor necrosis factor, antisense oligonucleotide, interference RNA, and combinations thereof

Therapeutic agents include but are not limited to, for example, chemotherapeutic drugs such as vinca alkaloids, anthracyclines, epidophyllotoxins, taxanes, antimetabolites, alkylating agents, antibiotics, antimitotics, antiangiogenic and proapoptotic agents, particularly doxorubicin, methotrexate, taxol, CPT-11, SN-38, camptothecans, and others from these and other classes of anticancer agents, and the like. Other cancer chemotherapeutic drugs include nitrogen mustards, alkyl sulfonates, nitrosoureas, triazenes, folic acid analogs, pyrimidine analogs, purine analogs, platinum coordination complexes, hormones, and the like. Suitable chemotherapeutic agents are described in Remington's Pharmaceutical compositions, 19th Ed. (Mack Publishing Co. 1995), and in Goodman and Gilman's the Pharmacological Basis of Therapautics, 7th Ed. (MacMillan Publishing Co. 1985), as well as revised editions of these publications. Other suitable chemotherapeutic agents, such as experimental drugs, are known to those of skill in the art, and may be conjugated to the bioactive assemblies described herein using methods that are known in the art.

Exemplary therapeutic peptides or proteins may include but is not limited to, for example: adrenocorticotropic hormone (ACTH); adrenocorticotropic hormone derivatives (e.g., ebiratide); angiotensin; angiotensin II; asparaginase; atrial natriuretic peptides; atrial sodium diuretic peptides; bacitracin; beta-endorphins; blood coagulation factors VII, VIII and IX; blood thymic factor (FTS); blood thymic factor derivatives (see U.S. Pat. No. 4,229,438); bombesin; bone morphogenic factor (BMP); bone morphogenic protein; bradykinin; caerulein; calcitonin gene related polypeptide (CGRP); calcitonins; CCK-8; cell growth factors (e.g., EGF; TGF-alpha; TGF-beta; PDGF; acidic FGF; basic FGF); cerulein; chemokines; cholecystokinin; cholecystokinin-8; cholecystokinin-pancreozymin (CCK-PZ); colistin; colony-stimulating factors (e.g. CSF; GCSF; GMCSF; MCSF); corticotropin-releasing factor (CRF); cytokines; desmopressin; dinorphin; dipeptide; dismutase; dynorphin; eledoisin; endorphins; endothelin; endothelin-antagonistic peptides (see European Patent Publication Nos. 436189; 457195 and 496452 and Japanese Patent Unexamined Publication Nos. 94692/1991 and 130299/1991); endotherins; enkephalins; enkephalin derivatives (see U.S. Pat. No. 4,277,394 and European Patent Publication No. 31567); epidermal growth factor (EGF); erythropoietin (EPO); follicle-stimulating hormone (FSH); gallanin; gastric inhibitory polypeptide; gastrin-releasing polypeptide (GRP); gastrins; G-CSF; glucagon; glutathione peroxidase; glutathio-peroxidase; glutaredoxin; gonadotropins (e.g., human chorionic gonadotrophin and .alpha. and .beta. subunits thereof); gramicidin; gramicidines; growth factor (EGF); growth hormone-releasing factor (GRF); growth hormones; hormone releasing hormone (LHRH); human artrial natriuretic polypeptide (h-ANP); human placental lactogen; insulin; insulin-like growth factors (IGF-I; IGF-II); interferon; interferons (e.g., alpha- beta- and gamma-interferons); interleukins (e.g. 1; 2; 3; 4; 5; 6; 7; 8; 9; 10; 11 and 12); intestinal polypeptide (VIP); kallikrein; kyotorphin; luliberin; luteinizing hormone (LH); luteinizing hormone-releasing hormone (LH-RH); lysozyme chloride; melanocyte-stimulating hormone (MSH); melanophore stimulating hormone; mellitin; motilin; muramyl; muramyldipeptide; nerve growth factor (NGF); nerve nutrition factors (e.g. NT-3; NT-4; CNTF; GDNF; BDNF); neuropeptide Y; neurotensin; oxytocin; pancreastatin; pancreatic polypeptide; pancreozymin; parathyroid hormone (PTH); pentagastrin; polypeptide YY; pituitary adenyl cyclase-activating polypeptides (PACAPs); platelet-derived growth factor; polymixin B; prolactin; protein synthesis stimulating polypeptide; PTH-related protein; relaxin; renin; secretin; serum thymic factor; somatomedins; somatostatins derivatives (Sandostatin; see U.S. Pat. Nos. 4,087,390; 4,093,574; 4,100,117 and 4,253,998); substance P; superoxide dismutase; taftsin; tetragastrin; thrombopoietin (TPO); thymic humoral factor (THF); thymopoietin; thymosin; thymostimulin; thyroid hormone releasing hormone; thyroid-stimulating hormone (TSH); thyrotropin releasing hormone TRH); trypsin; thyroidoxin; tuftsin; tumor growth factor (TGF-alpha); tumor necrosis factor (TNF); tyrocidin; urogastrone; urokinase; vasoactive intestinal polypeptide; vasopressins, and functional equivalents of such polypeptides.

Kits

Various embodiments may concern kits containing components suitable for assessing longevity in a subject or sample. Exemplary kits may contain at least one longevity-predicting marker complex. If the composition containing components for administration is not formulated for delivery via the alimentary canal, such as by oral delivery, a device capable of delivering the kit components through some other route may be included. One type of device, for applications such as parenteral delivery, is a syringe that is used to inject the composition into the body of a subject. Inhalation devices may also be used.

The kit components may be packaged together or separated into two or more separate containers. In some embodiments, the containers may be vials that contain sterile, lyophilized formulations of a composition that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers. Another component that can be included is instructions to a person using a kit for its use.

In one particular embodiment, a kit may include components for a longevity predicting marker complex of an alpha B crystallin-like sequence linked to a reporter. In a more particular embodiment, a kit may include components for a longevity-predicting marker complex of HSP-27 or HSP-27-like sequence linked to a reporter agent for measuring longevity and or health in a subject or a sample.

In one exemplary product, a kit could contain materials for sample collection such as a sterile disposable protective system (glove, small mask, disinfectant, gauze, cotton and bandage); sterile disposable container with label, including spare one (container may vary depending on type of specimen collected: blood, urine, tissue, hair, secretions); sterile disposable tool to collect the sample, including spare one (this may vary too depending on specimen: syringe, scraper, stick . . . ). Instructions for use (e.g. indication, side effects, warnings . . . ). Buffer or reagents necessary for the detection reaction (varies depending on specimen: protein, DNA, RNA); Probe with control “staining” maybe with different ranges (e.g. calorimetric pH stick or fluorescence detector stick): positive control (e.g. serum containing anti-HSP27, other alpha-B crystallin antibodies or aptamers; or similarly specific recognition system) and negative (e.g. preimmune serum). In accordance with this example, a secondary antibody or other detection system known in the art can be used to measure the presence of or level of a longevity predicting marker such as HSP27 or other alpha-B crystallin peptides, proteins or nucleic acid levels. See list of HSP27 assessment protocols.

The embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### EXAMPLES

### Example 1

Annual Checkup

In one exemplary method, a healthy 34 year-old female subject goes to her healthcare provider for her annual checkup. The healthcare provider takes a blood sample. In addition to standard blood tests, such as thyroid analysis, cholesterol analysis and liver analysis, the sample is tested for levels of a longevity predicting marker such as HSP27 expression (e.g. peptide, protein or nucleic acid levels). In addition, other related protein family members such as alpha-B crystallin protein may be screened for level of expression. These levels of expression are used to assess the likelihood that the patient will have a serious illness in the next period of time. The time of the next check up and types of therapies are based in part on her HSP27 status.

A first annual examination using the assessment of construct levels in a blood or buccal swab can be used as a base for comparison at the next examination. A dramatic change in levels of a longevity predicting marker, for example a HSP27 or another alpha B crystallin-like molecule, could be an indication of onset of serious illness such as a cardiac condition or cancer. In one example, a sudden change in serum level can be a prognostic of a tumor somewhere in the patient. This observation would be indicative of the requirement for further physical analysis on the patient. For example, an aggressive examination of the patient for the type and site of the tumor should be performed.

### Example 2

Annual Checkup; High Inducible Levels.

In one exemplary method, a healthy 45 year old male subject goes to his healthcare provider for his annual checkup. The healthcare provider takes a blood sample or a buccal swab. In addition to standard tests, such as thyroid analysis, cholesterol analysis and liver analysis, the sample or swab would be tested for levels of a longevity predicting marker such as HSP27 expression (e.g. peptide, protein or nucleic acid levels) and other related protein family members such as alpha-B crystallin protein. The level of expression could be used to assess the likelihood that the patient will have a serious illness in the next period of time. The time of the next check up and types of therapies are based in part on his HSP27 status. The patient presents with overall high levels of HSP27 induction which are associated with positive long-term survival. The time of the next checkup and types of therapies are based in part on his HSP27 status and his overall test results and health. These test results along with other positive standard test results indicate that the patient does not require any further work up at the time.

This is an assessment conducted on a healthy patient and utilizes fresh samples from the patient each time the patient visits his healthcare provider.

### Example 3

In one exemplary method, the literature suggests an inverse relationship between HSP-27 and cancer stage and progression for a number of cancer types. A 62 year-old male cancer patient presents with a cancer, such as lung cancer. The healthcare provider measures the levels of HSP27 systemically through for example, a blood sample, as well as a biopsy of the lung tumor. The level of aggressiveness of the subsequent treatment is based on his HSP27 level, which is a measure both of his systemic ability to fight cancer and his ability to withstand the cancer treatment. A lower level of HSP27 systemically would indicate the need to assess a more aggressive treatment of the tumor. A higher level of HSP27 in the tumor would also support a more aggressive therapy needed for the patient to attack the tumor cells. This examination combined with standard examinations of a cancer patient would provide a valuable estimate for increased chance of survival of the patient based on a more accurate assessment of the patient's overall health and well-being.

### Example 4

In one exemplary method a diagnostic assessment of physiology or “Physiologic Space” of an organism is examined. This concept is premised on the concept that the organism contains systems that are capable of self diagnosis and response to this diagnosis in terms of self regulative processes. These self-regulative processes monitor and determine metabolic state and a variety of other responses to environmental stressors, as well as to internal stresses.

Physiologic space was assessed in the nematode worm, C. elegans, to demonstrate that there are key outputs at the protein level which can be assessed externally and which are tightly related to the internal state of the organism. In this case, the detected response of HSP-16 was tightly correlated with a state of readiness of the cell and thus with its subsequent abilities to fight off metabolic disruption and pathogenic attack from both external and internal sources. Altering this metabolic state should therefore lead to increased capability to fight exogenous and endogenous attack. This state is associated with numerous molecular responses totaling hundreds, if not thousands of distinct protein and non-protein molecules modulated by the organism in its need to survive.

The evidence indicates that this physiologic state is responsible for heterogeneity in the population, both hidden and observed. Altering this physiology with treatments designed to modulate it can lead to dramatic gains in health outcomes for some individuals and for negative effects for others. The output marker HSP-16 is an assessment of this ability of the organism to deal with these stressors and to subsequently respond in such a way as to increase viability or alternatively increased susceptibility and frailness.

In one exemplary method, when both genotype and environment are held constant, “chance” variation in the lifespan of individuals in a population is still quite large. Using isogenic populations of the nematode Caenorhabditis elegans, on the first day of adult life, chance variation in the level of induction of a green fluorescent protein (GFP) reporter coupled to a promoter from the hsp-16.2 gene, predicted as much as a four-fold variation in subsequent survival. The same reporter is also a predictor of ability to withstand a subsequent lethal thermal stress. The level of induction of GFP is not heritable and GFP expression levels in other reporter constructs are not associated with differential longevity.

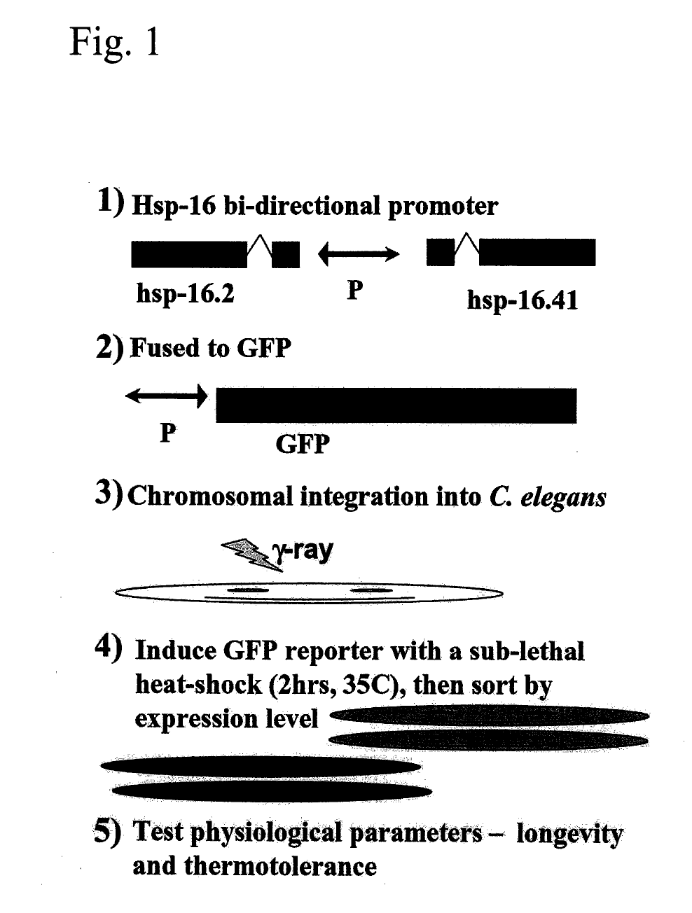

In this example, the optical transparency of C. elegans allows non-invasive visual assessment of living worms without compromising subsequent measurement of longevity. Chromosomally-integrated transgenic strain (TJ375), containing the 400 bp hsp-16.2 promoter coupled to the gene encoding green fluorescent protein (GFP) and encoding no HSP-16.2 product itself (FIG. 1). This reporter provides an accurate assessment of the total amount of native HSP-16.2 protein. No detectable GFP is observed in uninduced worms, but following a one or two-hour 35° C. pulse, GFP becomes readily apparent, peaking at 15-18 hours (data not shown).

Heat-shocked populations displayed a wide and near normally-distributed variation in individual GFP fluorescence (data not shown), even though individuals were isogenic and grown in an environment designed to minimize environmental heterogeneity. Such heterogeneity was observed from the earliest times at which GFP expression was detectable and continued until the time GFP had completely dissipated (several days, data not shown). The degree of heterogeneity increased markedly with time (data not shown), and was both replicable and quantifiable (data not shown).

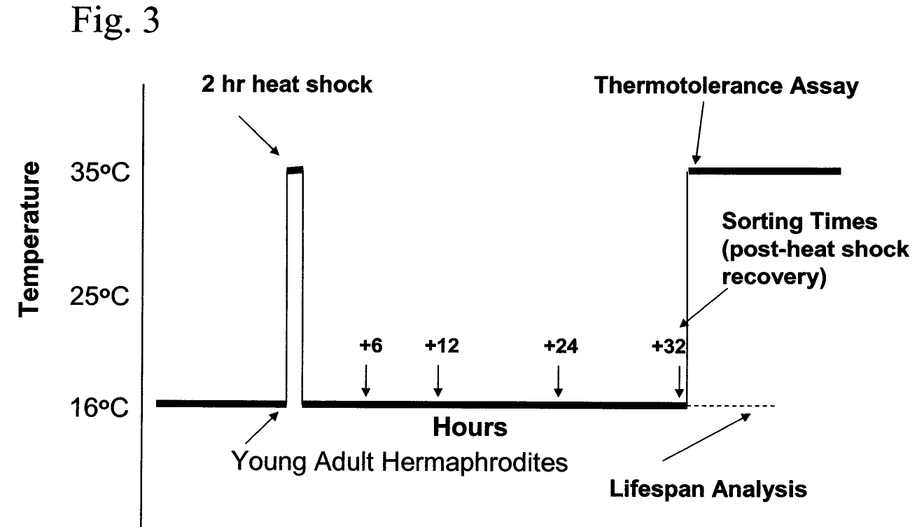

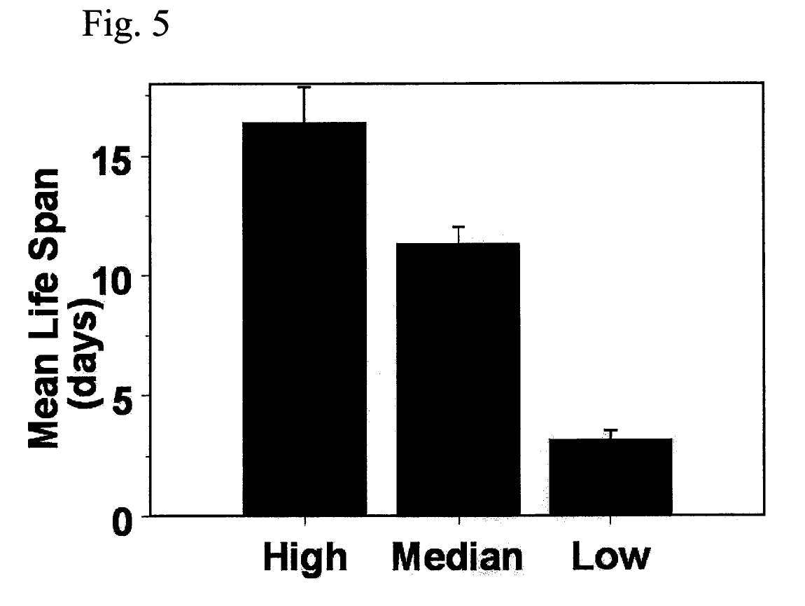

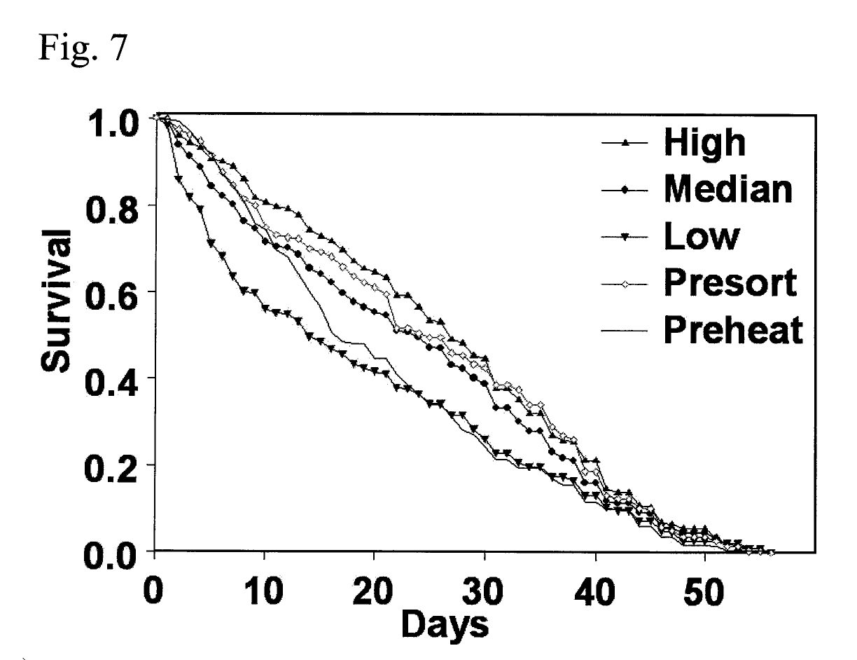

HSP-16::GFP expression was analyzed to assess whether the expression might predict longevity (FIG. 5). In isogenic worms measured manually, recorded data suggested a significant correlation between GFP-expression level and subsequent longevity (r =0.48; P =0.002); so we extended our studies to large populations. Worms were sorted into high, intermediate, and low GFP-expression classes at various times after heat induction (FIG. 3) and were subsequently tested for resistance to a lethal thermal stress or kept for longevity analysis. It was observed that significant differences in subsequent longevity and thermotolerance among worms that expressed GFP at high, average or low levels (FIGS. 5 and 7). When sorted after a two-hour induction at 35° C., worms that differentially expressed GFP showed large differences in life expectancy and thermotolerance. In a typical experiment, we found life expectancies of 16.4 days in the brightest worms, while the worms expressing the lowest levels of GFP after heat shock lived only about 3.2 days (FIG. 5). Similarly, worms with the highest GFP levels also showed higher thermotolerance (9.5 hours) than the average (6.7 hours) or than worms showing the lowest levels of thermotolerance (4.0 hours; FIG. 2d; P <0.001).

Samples were sorted at different times after the heat shock to observe differential survival. Differential life expectancy was found to vary as much as 10 to 15 days (example in FIG. 3), averaging 8.0 days over all 19 experiments (data not shown). Following two-hours of induction, lifespan averaged 15.1 days for the high and only 7.1 days for the low over all experiments, more than a two-fold difference (data not shown; P=8.0×10⁻²⁹). Sorting earlier than 9 hours after heatshock led to non-significant results. Worms expressing different levels of GFP, 9 to 36 hours after induction, also differed significantly with respect to subsequent thermotolerance (data not shown). Statistically significant differences were demonstrated. This differential thermotolerance was very robust, averaging about 3.4 hours (data no shown; P=1.7×1 0⁻²⁸). Differential survival and thermotolerance were highest at about 18 hours after induction, about the time when variance is maximal.

To correct for possible interrelationships between the effect of heat on survival and its effect on fertility, two temperature-sensitive (ts) fertility mutations, fer-15(b26) and spe-9(hc88), were crosses into the TJ375 reporter strain to form a new strain: TJ550. At the non-permissive temperature, the combination of both mutations completely blocked reproduction, but not germ cell formation or proliferation⁵. Differential survival was, however, still observed in this background with all six replicates showing significant differences in longevity between bright and dim worms (data not shown).

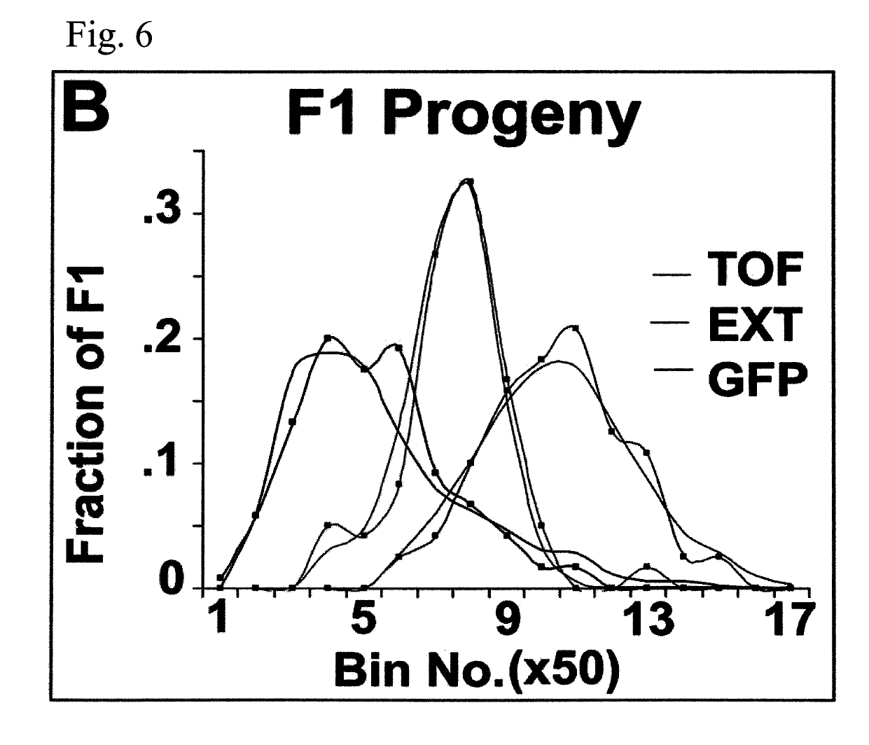

Individual differences in hsp-16.2:: GFP reporter expression may result from genetic variation—i.e. epigenetic changes may occur in isogenic individuals during propagation leading to differential inactivation or expression of one or more of the large number of repeats in the transgenic array present in the reporter strains. To address this question, differences in levels of GFP expression were analyzed for heritability. A population was sorted at 11 hours post heat shock into sub-populations containing a few hundred of the total initial population of 60,000 worms (FIG. 6). Progeny were collected, allowed to grow to maturity, induced by heat shock and assessed for level of GFP expression using the identical protocol. It was found that progeny of both the high- and low-expressing parents showed almost identical average levels of GFP expression (FIG. 6). Progeny of both high- and low-expression parental classes had almost the same mean expression level (298.0 vs 288.6 GFP units) and both displayed almost identical variation in levels of GFP expression (p=0.5, X² test for distributional difference), essentially recapitulating that of the parental population. Thus, the precise level of GFP expression is not heritable. While it is possible that further experimentation may reveal discrete causal factors determining variance of GFP expression, the results shown here were obtained from an isogenic population, maintained in a uniform environment during their propagation. Non-heritability of GFP expression level suggests the presence of a large underlying stochastic component specifying level of GFP expression in individual worms, similar to that observed in bacteria.

Finally, the level of GFP fluorescence was assessed as a predictor of longevity when GFP is tagged to promoters of non stress-inducible genes (myo-2 and mtl-2). It is not. Since GFP fluorescence is dependent upon redox activation we also utilized a promoter tag of a gene normally activated in response to oxidative stress (gst-4) and again found no relationship between GFP levels and subsequent longevity (FIG. 7).

HSP-16.2 expression level in young adults is a robust predictor of remaining life expectancy. This variation is not heritable. Even under rigidly controlled laboratory conditions, 60% of the variation in longevity in F₂ intercrosses in nematodes is not genetic. Similar findings are true in all species that have been studied; in humans, only about 25% of the variation in life span (even after excluding early deaths due to childhood disease and accident), is due to measurable genetic effects leaving the vast majority of variation in life span as unexplained or “environmental”, some of which results from chance or stochastic events within individuals.

Stochastic variation arises from fundamental thermodynamic and statistical mechanical considerations. A large fraction of individual variation in life span must stem from the fundamental fact that life results from an integrated series of metabolic reactions which themselves are under fundamental physical constraints on the specificity and rigidity with which they, too, can be regulated. At the molecular level, two points are germane to the present study. First, when the number of molecules regulating a biological process becomes countably small, “chance” distributions come into play such that some regulatory molecules can vary several-fold between individual cells. Second, the Maxwell-Boltzmann (M-W) equation specifies the distribution of kinetic energies among molecules and requires kinetic energy to be a distributed function. This equation was utilized to develop a general theory explaining mortality kinetics. Several sources of variation at the molecular level could conceivably alter GFP (HSP-16.2) expression level while simultaneously affecting more global processes. These include intracellular differences and fluctuations in the rates of molecular processes such as transcription, ribosome loading and translation as previously examined. Chance variation in the number of HSF effector molecules present within each cell at the time of heat shock also could have dramatic phenotypic consequences. Variation in the frequency of mitochondrial genomic rearrangements, as previously observed in isogenic populations of C. elegans, could have an effect. There is an increasing literature describing variation among isogenic individuals at the molecular level, typically in microbial or yeast cultures where such effects can be visualized. Clearly, significant variation among genetically identical individuals is a fact of nature and inherent molecular variability implies that biochemical and molecular genetic processes must exhibit inherent variability.

A Biomarker of Aging (BoA) has been defined as a biological parameter of an organism that either alone or in some multivariate composite will, in the absence of disease, better predict functional capability at some later age than will chronological age. These studies support that level of HSP-16 production may now also be such a BoA and that it is a robust predictor of subsequent individual longevity. It seems likely that the hsp-16.2::GFP reporter is conveying information about the general physiological state of the cell and/or organism with respect to its ability to withstand stress and its subsequent likelihood of survival.

All of the COMPOSITIONS and/or METHODS and/or APPARATUS disclosed and claimed herein can be made and executed without undue experimentation in light of the present disclosure. While the compositions and methods of this invention have been described in terms of preferred embodiments, it will be apparent to those of skill in the art that variation may be applied to the COMPOSITIONS and/or METHODS and/or APPARATUS and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of use comprising:
a) obtaining at least one sample from a subject;
b) determining the level of one or more longevity predicting marker(s) in the sample(s); and
b) predicting over all health of a subject based the marker level.

2. The method of claim 1, wherein the longevity predicting marker is selected from the group consisting of alpha-B crystallin, alpha A-crystallin, alpha-B crystallin-like, alpha A-crystallin-like molecules and a combination thereof.

3. The method of claim 2, wherein the alpha-B crystallin-like molecule is a heat shock protein 27 (HSP27), a HSP27 analog, a HSP27 derivative, a HSP27 peptide molecule, a HSP27 nucleic acid molecule or combination thereof.

4. The method of claim 1, wherein the subject is human.

5. The method of claim 1, wherein the sample comprises at least one of a urine, blood, tissue, saliva, amniotic fluid, cerebrospinal fluid, tears or combination thereof.

6. The method of claim 1, wherein the marker is a biomarker of aging (BoA).

7. The method of claim 1, wherein the marker level presence or level is determined by ELISA, Western blot, immunoassay, oligonucleotides probe hybridization, microarray analysis or sequence specific amplification.

8. The method of claim 1, wherein the marker level is determined in one or more cell lines isolated from the subject.

9. The method of claim 8, wherein the cell line is homogeneous or inhomogeneous.

10. The method of claim 9, wherein the level of an endogenous marker is determined in the cell line.

11. The method of claim 9, wherein the level of a transgenic marker is determined in the cell line.

12. The method of claim 1, wherein a greater level of one or more longevity predicting marker(s) in the sample(s) is predictive of better overall health.

13. The method of claim 1, wherein a lower level of one or more longevity predicting marker(s) in the sample(s) is predictive of the presence or onset of a condition.

14. The method of claim 13, wherein the condition is selected from the group consisting of cardiac condition, cancerous condition, autoimmune condition, liver condition, kidney condition, infectious condition, eye condition, infertility condition, neural condition or combination thereof.

15. The method of claim 1, further comprising determining the need for other physical assessment tests of the subject based on the level(s) of marker(s) in the sample.

16. A method of use comprising:
a) obtaining at least one sample from a subject;
b) determining the level of one or more longevity predicting marker(s) in the sample(s); and
b) predicting longevity of a subject based the marker level(s).

17. The method of claim 16, wherein the subject is a human.

18. The method of claim 16, wherein the subject is a cow, a chicken, a goat, a horse, a pig, a sheep, a cat, a dog, an alpacas, a fish, or a bird.

19. A method of use comprising:
a) treating a subject with a potential therapeutic intervention;
b) determining the effect of the treatment on the level of a longevity predicting marker; and
b) using the marker response to predict the effectiveness of the intervention.

20. A method of use comprising:
a) obtaining at least one sample from a subject;
b) determining the level of one or more longevity predicting marker(s) in the sample(s);
c) treating a subject with a potential therapeutic intervention based on the level of longevity predicting marker(s) in the subject;
d) determining the effect of the therapeutic intervention on the level of a longevity predicting marker; and
e) using the marker response to predict the effectiveness of the intervention.

21. The method of claim 20, wherein the therapeutic intervention comprises cardiac therapy, cancer therapy, autoimmune therapy, hepatic treatment, kidney therapy, anti-bacterial therapy, anti-viral therapy, anti-fungal therapy, eye therapy, infertility therapy, neural therapy or combination thereof.
